(19) United States
(12) Patent Application Publication
(10) Pub. No.: US 2020/0265940 A1
(43) Pub. Date: Aug. 20, 2020

(54) PREDICTIVE TREATMENT CONTROLLER FOR VEDOLIZUMAB

(71) Applicant: The Regents of the University of California, Oakland, CA (US)

(72) Inventor: Parambir Dulai, San Diego, CA (US)

(21) Appl. No.: 16/792,090

(22) Filed: Feb. 14, 2020

Related U.S. Application Data
(60) Provisional application No. 62/806,639, filed on Feb. 15, 2019.

(51) Int. Cl. G16H 20/17; G01N 33/577; G16H 50/50; G16H 50/30; G16H 10/60; G16H 50/70; G06N 5/04; G06N 20/00

(52) U.S. Cl. CPC G16H 20/17; G01N 33/577; G16H 50/50; G16H 50/30; G01N 2800/52; G16H 50/70; G06N 5/04; G06N 20/00; G16H 10/60

(57) ABSTRACT

A method for predicting an outcome of treating an ulcerative colitis patient with vedolizumab may include applying a predictive model to determine a response indicator indicative of the outcome of treating the ulcerative colitis patient with vedolizumab. The predictive model may be based on a plurality of predictive factors including a duration of disease, an exposure to tumor necrosis factor antagonist therapy, a baseline endoscopy corresponding to a disease severity, and/or a concentration of albumin. The response indicator may include a probability of the ulcerative colitis patient responding to vedolizumab, achieving a clinical remission and/or an endoscopic remission of ulcerative colitis with vedolizumab, requiring surgical intervention, and/or encountering an infection when treated with vedolizumab. A treatment plan including a dose schedule for administering vedolizumab to the ulcerative colitis patient may be determined based on the response indicator.

### CROSS REFERENCE TO RELATED APPLICATION

This application claims priority to U.S. Provisional Application No. 62/806,639, which is filed on Feb. 15, 2019 and entitled “PREDICTION TOOL FOR VEDLIZUMAB DRUG EXPOSURE AND EFFICACY,” the disclosure of which is incorporated herein by reference in its entirety.

### TECHNICAL FIELD

The subject matter described herein relates generally to the treatment of ulcerative colitis and more specifically to techniques for predicting the safety and efficacy of vedolizumab as a treatment for ulcerative colitis.

### BACKGROUND

Ulcerative colitis is an inflammatory bowel disease (IBD) affecting the innermost lining of the large intestine (colon) and rectum. A chronic condition, ulcerative colitis may be the result of various factors including, for example, abnormal immune response, genetics, microbiome, and environmental triggers. Sores (e.g., ulcers) may develop in the lining of the large intestine and rectum as a result of ulcerative colitis.

### SUMMARY

Systems, methods, and articles of manufacture, including computer program products, are provided for predicting the outcome of an ulcerative colitis treatment that includes vedolizumab. In one aspect, there is provided a system that includes at least one data processor and at least one memory. The at least one memory may store instructions that result in operations when executed by the at least one data processor. The operations may include: applying a predictive model to determine a response indicator indicative of the outcome of treating the ulcerative colitis patient with vedolizumab, the response indicator including a first probability of the ulcerative colitis patient responding to vedolizumab, the predictive model based on a plurality of predictive factors including a duration of disease; and determining, based at least on the response indicator, a treatment plan for the ulcerative colitis patient.

In some variations, one or more features disclosed herein including the following features may optionally be included in any feasible combination. The response indicator may further include a second probability of the ulcerative colitis patient achieving a clinical remission and/or an endoscopic remission of ulcerative colitis with vedolizumab, a third probability of the ulcerative colitis patient achieving a rapid response to vedolizumab, a fourth probability of the ulcerative colitis patient requiring surgical intervention, and/or a fifth probability of infection when treated with vedolizumab.

In some variations, the plurality of predictive factors may further include an exposure to tumor necrosis factor antagonist therapy.

In some variations, the plurality of predictive factors may further include a baseline endoscopy corresponding to a disease severity.

In some variations, the plurality of predictive factors may further include a concentration of albumin.

In some variations, the duration of disease may include a first value to indicate a duration that is less than a quantity of time or a second value to indicate a duration that is equal to or greater than the quantity of time.

In some variations, the predictive model may be generated by at least identifying, based on one or more observational datasets associated with vedolizumab, the plurality of predictive factors. The identifying of the plurality of predictive factors may include generating a first cohort of ulcerative colitis patients. The first cohort of ulcerative colitis patients may be generated by at least removing, from the one or more observational datasets, data associated with ulcerative colitis patients exposed to a placebo instead of vedolizumab.

In some variations, the generating of the predictive model may further include generating a second cohort of ulcerative colitis patients. A performance of the predictive model is verified based at least on data associated with the second cohort of ulcerative colitis patients.

In some variations, the generating of the predictive model may further include excluding, from the predictive model, a first predictive factor that exhibits a co-linearity with a second predictive factor.

In some variations, the generating of the predictive model may further include determining, for each of the plurality of predictive factors, a weight corresponding to a correlation to a treatment outcome of vedolizumab.

In some variations, the plurality of predictive factors may be identified by performing a logistic regression on the one or more observational datasets to identify the plurality of predictive factors as having an above-threshold correlation with a treatment outcome of vedolizumab.

In some variations, the treatment plan for the ulcerative colitis patient may include vedolizumab in response to the response indicator exceeding a threshold value.

In some variations, the treatment plan for the ulcerative colitis patient may further include a dose quantity comprising a quantity of vedolizumab administered in each dose of vedolizumab. The treatment plan for the ulcerative colitis patient may further include a dose schedule for administering vedolizumab. The dose schedule may include a length of an interval between successive doses of vedolizumab. The length of the interval between successive doses of vedolizumab may correspond to the response indicator associated with the ulcerative colitis patient. The ulcerative patient may be treated in accordance with the treatment plan including by being administered the quantity of vedolizumab at one or more intervals indicated by the dose schedule.

In another aspect, there is provided a method for predicting the outcome of an ulcerative colitis treatment that includes vedolizumab. The method may include: applying a predictive model to determine a response indicator indicative of the outcome of treating the ulcerative colitis patient with vedolizumab, the response indicator including a first probability of the ulcerative colitis patient responding to vedolizumab, the predictive model based on a plurality of predictive factors including a duration of disease; and determining, based at least on the response indicator, a treatment plan for the ulcerative colitis patient.

In some variations, one or more features disclosed herein including the following features may optionally be included in any feasible combination. The response indicator may further include a second probability of the ulcerative colitis patient achieving a clinical remission and/or an endoscopic remission of ulcerative colitis with vedolizumab, a third probability of the ulcerative colitis patient achieving a rapid response to vedolizumab, a fourth probability of the ulcerative colitis patient requiring surgical intervention, and/or a fifth probability of infection when treated with vedolizumab.

In some variations, the plurality of predictive factors may further include an exposure to tumor necrosis factor antagonist therapy, a baseline endoscopy corresponding to a disease severity, and/or a concentration of albumin.

In another aspect, there is provided a computer program product including a non-transitory computer readable medium storing instructions. The instructions may cause operations when executed by at least one data processor. The operations may include: applying a predictive model to determine a response indicator indicative of the outcome of treating the ulcerative colitis patient with vedolizumab, the response indicator including a first probability of the ulcerative colitis patient responding to vedolizumab, the predictive model based on a plurality of predictive factors including a duration of disease; and determining, based at least on the response indicator, a treatment plan for the ulcerative colitis patient.

Implementations of the current subject matter can include, but are not limited to, systems and methods consistent including one or more features are described as well as articles that comprise a tangibly embodied machine-readable medium operable to cause one or more machines (e.g., computers, etc.) to result in operations described herein. Similarly, computer systems are also described that may include one or more processors and one or more memories coupled to the one or more processors. A memory, which can include a computer-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein may be apparent from the description and drawings, and from the claims. While certain features of the currently disclosed subject matter are described for illustrative purposes in relation to treating ulcerative colitis with vedolizumab, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating a vedolizumab treatment analysis system, in accordance with some example embodiments;

FIG. 2A depicts a table illustrating the demographics of ulcerative patient cohorts, in accordance with some example embodiments;

FIG. 2B depicts a table illustrating an example of a predictive model, in accordance with some example embodiments;

FIG. 2C depicts a table illustrating a diagnostic performance of a predictive model, in accordance with some example embodiments;

FIG. 3A depicts a graph illustrating an outcome of treatment with vedolizumab, in accordance with some example embodiments;

FIG. 3B depicts a table illustrating a performance of the predictive model, in accordance with some example embodiments;

FIG. 3C depicts a table illustrating a performance of the predictive model, in accordance with some example embodiments;

FIG. 3D depicts a table illustrating a relationship between vedolizumab concentration and response, in accordance with some example embodiments;

FIG. 3E depicts a graph illustrating a relationship between vedolizumab concentration and response, in accordance with some example embodiments;

FIG. 3F depicts a graph illustrating a performance of the predictive model, in accordance with some example embodiments;

FIG. 3G depicts a graph illustrating a performance of the predictive model, in accordance with some example embodiments;

FIG. 4A depicts a flowchart illustrating an example of a process for determining responsiveness to vedolizumab, in accordance with some example embodiments;

FIG. 4B depicts a flowchart illustrating an example of a process for generating a predictive model, in accordance with some example embodiments; and

FIG. 5 depicts a block diagram illustrating a computing system, in accordance with some example embodiments.

When practical, similar reference numbers denote similar structures, features, or elements.

### DETAILED DESCRIPTION

Treatment for ulcerative colitis may include medication, dietary changes, and surgical intervention (e.g., colectomy). Vedolizumab (VDZ) is one example of a medication that may be used to treat ulcerative colitis. For example, ulcerative colitis patients treated with vedolizumab may achieve clinical remission, corticosteroid-free remission (CSFREM), and mucosal healing. Nevertheless, treatment outcome for vedolizumab may be variable. That is, not all ulcerative colitis patients treated with vedolizumab may achieve clinical response and/or clinical remission. For instance, in clinical practice, pooled rates for clinical response by week 22 of treatment was 51% (95% C1, 43-61%) and pooled rates for clinical remission by week 22 of treatment was 30%.

Accordingly, in some example embodiments, whether treatment for an ulcerative colitis patient should include vedolizumab may be determined based at least on a prediction of the outcome of subjecting the patient to a treatment that includes vedolizumab. A treatment controller may therefore be configured to generate, based on one or more predictive factors associated with clinical remission, corticosteroid-free remission (CSFREM), a prediction of the outcome of the treatment including vedolizumab. Examples of predictive factors may include exposure to prior tumor necrosis factor (TNF) antagonist therapy, duration of disease, disease severity (e.g., baseline endoscopy), albumin concentration in blood, and/or the like. The treatment controller may weigh each predictor to generate a response indicator indicative of the patient's response to a treatment including vedolizumab.

In some example embodiments, a higher response indicator may indicate a higher probability of responding to the treatment including vedolizumab, a higher probability of achieving a rapid response to the treatment including vedolizumab, a higher probability of achieving corticosteroid-free clinical or endoscopic remission, a lower probability of requiring surgical intervention (e.g., colectomy to remove the colon), and/or a lower probability of infection during the treatment including the vedolizumab. Alternatively, a lower response indicator may indicate a lower probability of responding to the treatment including vedolizumab, a lower probability of achieving a rapid response to the treatment including vedolizumab, a lower probability of achieving corticosteroid-free clinical or endoscopic remission, a higher probability of requiring surgical intervention (e.g., colectomy to remove the colon), and/or a higher probability of infection during the treatment including the vedolizumab. As such, whether the treatment for the ulcerative colitis patient should include vedolizumab may be determined based at least on the response indicator associated with the patient. For example, treatment for the ulcerative colitis patient may include vedolizumab if the response indicator associated with the patient exceeds a threshold value.

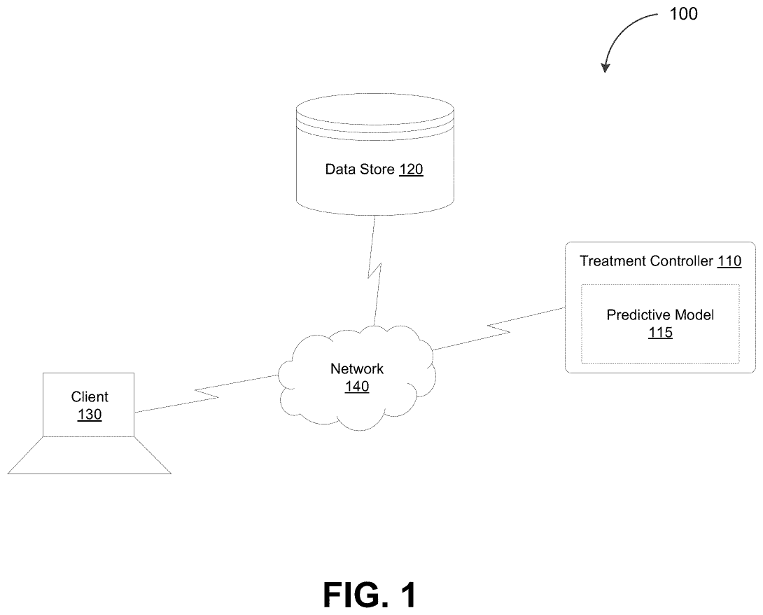

FIG. 1 depicts a system diagram illustrating a vedolizumab treatment analysis system 100, in accordance with some example embodiments. Referring to FIG. 1, the vedolizumab treatment analysis system 100 may include a treatment controller 110, a data store 120, and a client 130. The treatment controller 110, the data store 120, and the client 130 may be communicatively coupled via a network 140. The data store 120 may be a database including, for example, a relational database, a non-relational database, and/or the like. The client 130 may be a processor-based device including, for example, a workstation, a desktop computer, a laptop computer, a tablet computer, a mobile device, a wearable apparatus, and/or the like. The network 140 may be a wired network and/or a wireless network including, for example, a local area network (LAN), a virtual local area network (VLAN), a wide area network (WAN), a public land mobile network (PLMN), the Internet, and/or the like.

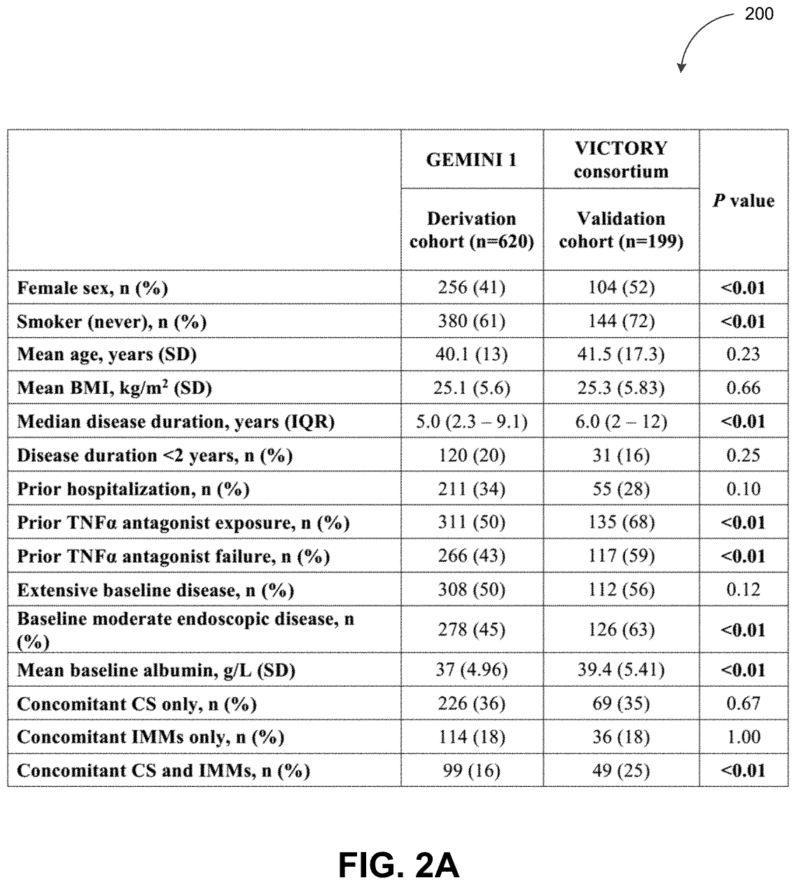

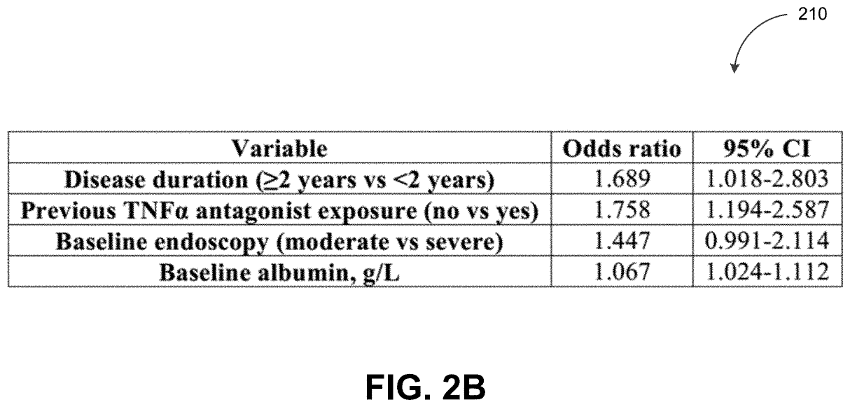

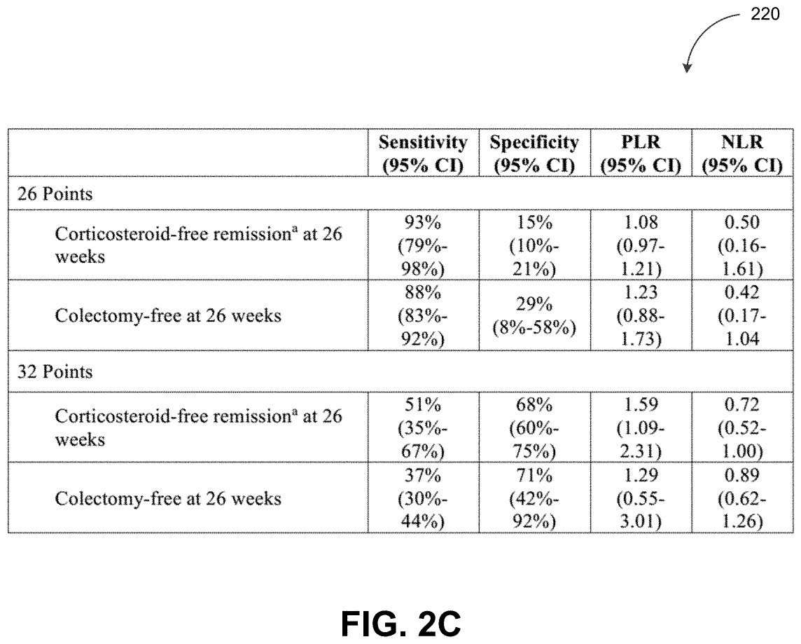

The treatment controller 110 may be configured to generate a predictive model 115 including by identifying, based on at least a portion of patient data stored in the data store 120, one or more predictive factors associated with clinical remission, corticosteroid-free remission (CSFREM). For example, the predictive factors forming the predictive model 115 may include prior tumor necrosis factor (TNF) antagonist therapy, duration of disease, level of endoscopy activity, albumin concentration in blood, and/or the like. Patient data in the data store 120 may include one or more observational datasets (e.g., clinical trial datasets and/or the like) associated with cohorts of ulcerative colitis patients who have been treated with vedolizumab. FIG. 2A depicts a table 200 depicting the demographics of ulcerative colitis patient cohorts, in accordance with some example embodiments. The observational datasets may include measured exposure to vedolizumab, onset of action, and overall efficacy and safety of the vedolizumab treatment. It should be appreciated that data associated with patients treated with a placebo may be excluded from the observational datasets. Moreover, as used herein, clinical remission, corticosteroid-free remission (CSFREM) may be defined as a full Mayo score of ≤2, with no subscore >1, and being off corticosteroids at 52 weeks. Alternatively, clinical remission, corticosteroid-free remission may be defined as achieving complete resolution of ulcerative colitis-related symptoms (e.g., rectal bleeding, urgency, stool frequency, and/or the like), a Mayo endoscopic subscore of 0 or 1, and being off corticosteroids at 26 weeks.

In some example embodiments, the predictive model 115 may be a multivariable logistic regression prediction model generated based on the patient data stored in the data store 120 with clinical remission, corticosteroid-free remission (CSFREM) as the dependent variable. Baseline variables with P value <0.15 on univariable analyses were included after assessment for co-linearity, clinical importance, and interpretability. A backward model selection approach with a P value threshold (e.g., a P value threshold of 0.15) for inclusion may be applied. Interaction terms may be assessed individually with interaction terms having a below threshold P value (e.g., P<0.10) on the univariable analysis as well as the multivariable analysis being included in the predictive model 115. A sensitivity analysis may be performed in which albumin may be replaced with calculated individual patient vedolizumab drug clearance profiles based on measured vedolizumab exposure to determine whether this modification is more predictive of clinical remission, corticosteroid-free remission (CSFREM).

The treatment controller 110 may determine to include, in the predictive model 115, one or more predictive factors having an above threshold association (e.g., P<0.15) with an increased probability of achieving clinical remission, corticosteroid-free remission (CSFREM) with a treatment including vedolizumab. These predictive factors may include disease duration (e.g., odds ratio, 1.04 per year), lack of previous tumor necrosis factor antagonist exposure (odds ratio, 1.84), no previous tumor necrosis factor antagonist therapy failure (odds ratio, 1.88), baseline endoscopic activity (moderate vs severe: odds ratio, 1.57), baseline stool frequency (nonsevere [partial Mayo score 0-2] vs severe [partial Mayo score 3]: odds ratio, 1.70), and baseline albumin (odds ratio, 1.08). However, it should be appreciated that one or more predictive factors may be transformed or excluded in order to optimize the predictive model 115. For example, disease duration may be transformed into a binary categorization (e.g., ≥x quantity of time and <x quantity of time) whereas previous tumor necrosis factor antagonist exposure may be used in lieu of previous tumor necrosis factor antagonist failure. To maximize objectivity, baseline endoscopy may be used as a metric for disease activity instead of less objective metrics such as stool frequency.

In some example embodiments, the predictive factors included in the predictive model 115 may include disease duration (e.g., ≥2 years and <2 years), previous tumor necrosis factor (TNF) antagonist exposure (e.g., no vs yes), baseline endoscopy (moderate vs severe), and baseline albumin (e.g., absolute value). To further illustrate, FIG. 2B depicts a table 210 illustrating an example of the predictive model 115, in accordance with some example embodiments.

At least some predictive factors may be excluded from the predictive model 115 in order to optimize the predictive model 115. For example, although gender exhibits a significant relationship to vedolizumab clearance (P<0.001), gender is nevertheless excluded from the predictive model 115 due to gender being an indirect predictor of treatment outcomes through correlation with the known covariates of drug regulatory clearance such as height and weight. Previous tumor necrosis factor (TNF) antagonist exposure and disease severity (e.g., baseline endoscopy) may be consistent predictors of reduced responsiveness to vedolizumab and are therefore included in the predictive model 115. Likewise, albumin concentration is included in the predictive model 115 because albumin concentration may be a main determinant of vedolizumab clearance. Contrastingly, although patients with longer disease duration are more likely to have been exposed to tumor necrosis factor antagonists before initiation of vedolizumab therapy and are therefore less likely to respond to vedolizumab therapy, patients with longer disease duration were nevertheless more likely to respond to vedolizumab. As such, disease duration remains a predictive factor in the predictive model 115.

The predictive model 115 may output a response indicator indicative of whether an ulcerative colitis patient treated with vedolizumab will respond to the treatment and achieve clinical remission, corticosteroid-free remission (CSFREM). The response indicator may be sent to the client 130, for example, displayed as part of a user interface associated with the treatment controller 110. For example, the client 130 may be associated with a healthcare provider and/or a health care payer. The response indicator displayed at the client 130 may be used to determine whether the treatment for the ulcerative colitis patient includes vedolizumab and/or an alternative treatment (e.g., medication, surgical intervention, and/or the like). In some example embodiments, the response indicator displayed at the client 130 may be further used to determine a dose quantity and/or a dose schedule for the ulcerative colitis patient including, for example, a length of interval between successive doses of vedolizumab. The response indicator displayed at the client 130 may be further used to determine whether the ulcerative colitis patient should be included in a study for alternative drugs and/or treatments.

Equation (1) below shows that the response indicator I may correspond to a weighed sum of the one or more predictive factors including, for example, disease duration (e.g., ≥2 years and <2 years), previous tumor necrosis factor (TNF) antagonist exposure (e.g., no vs yes), baseline endoscopy (moderate vs severe), and baseline albumin (e.g., absolute value). As shown in Equation (1), each predictive factor may be associated with a weight corresponding to a correlation to treatment outcome. For example, a first predictive factor may be associated with a higher weight than a second predictive factor if the first predictive factor exhibits a higher correlation to treatment outcome than the second predictive factor.

I=−3.7038+[0.2820 if no prior TNF antagonist exposure]+[0.2622 if disease duration>2 years]+[0.1847 if baseline endoscopy activity is moderate]+[0.0647 x baseline albumin concentration ing/L] (1)

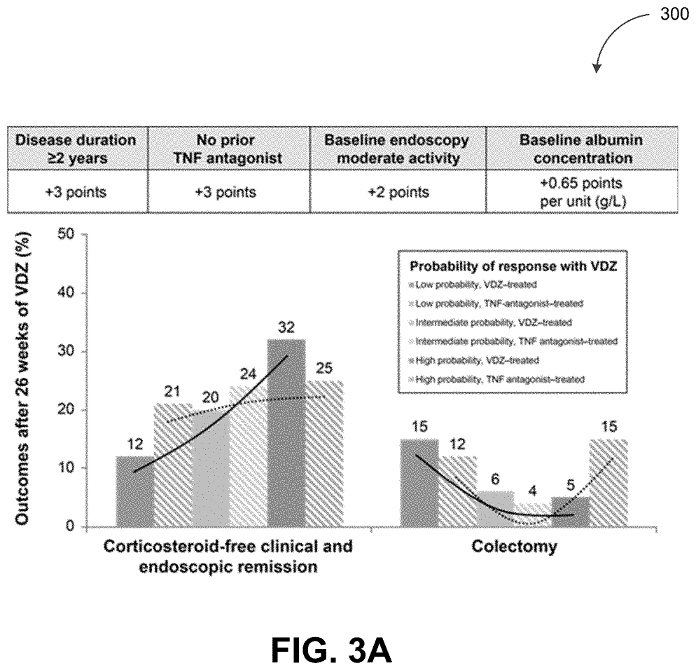

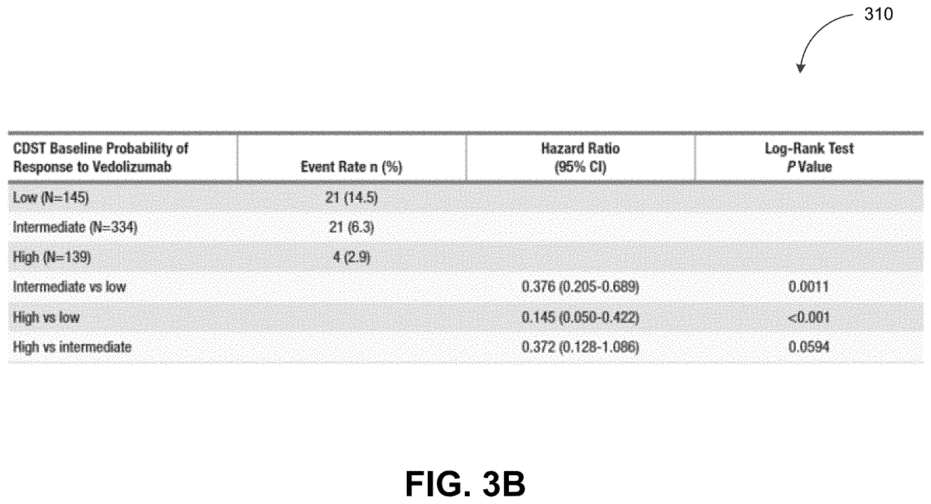

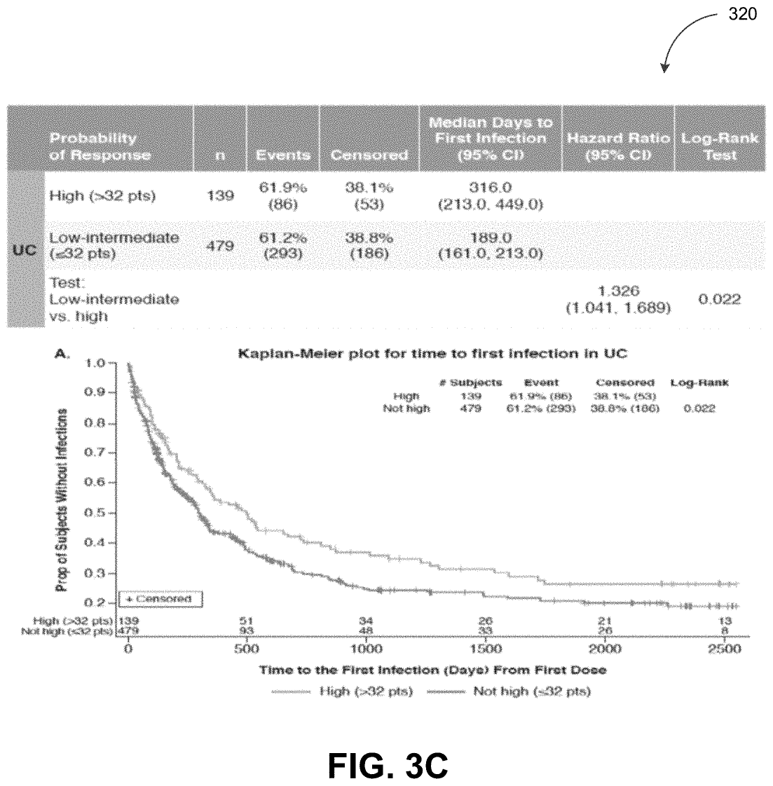

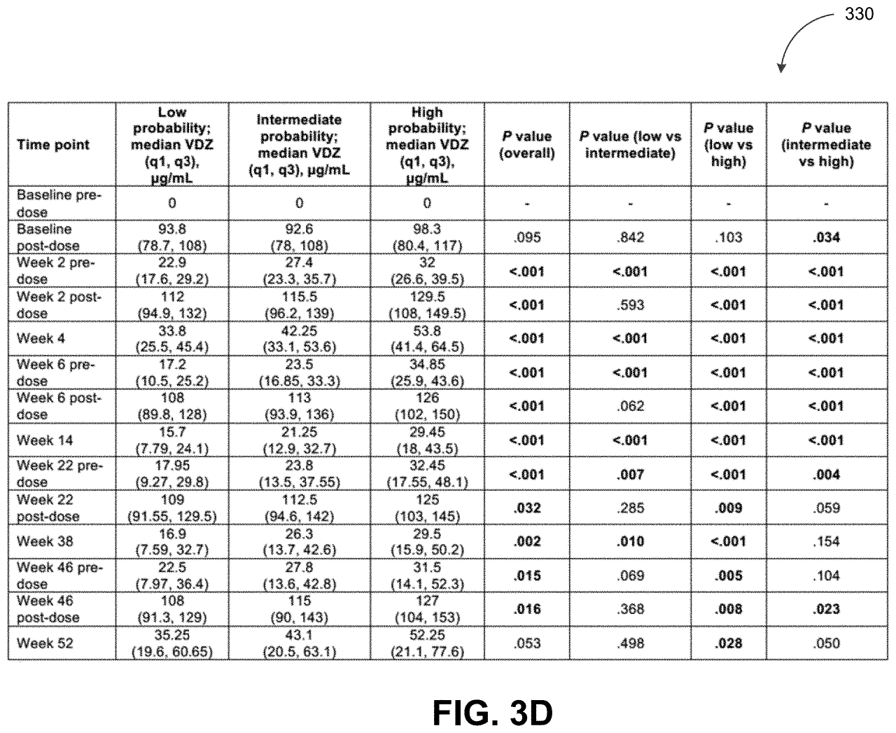

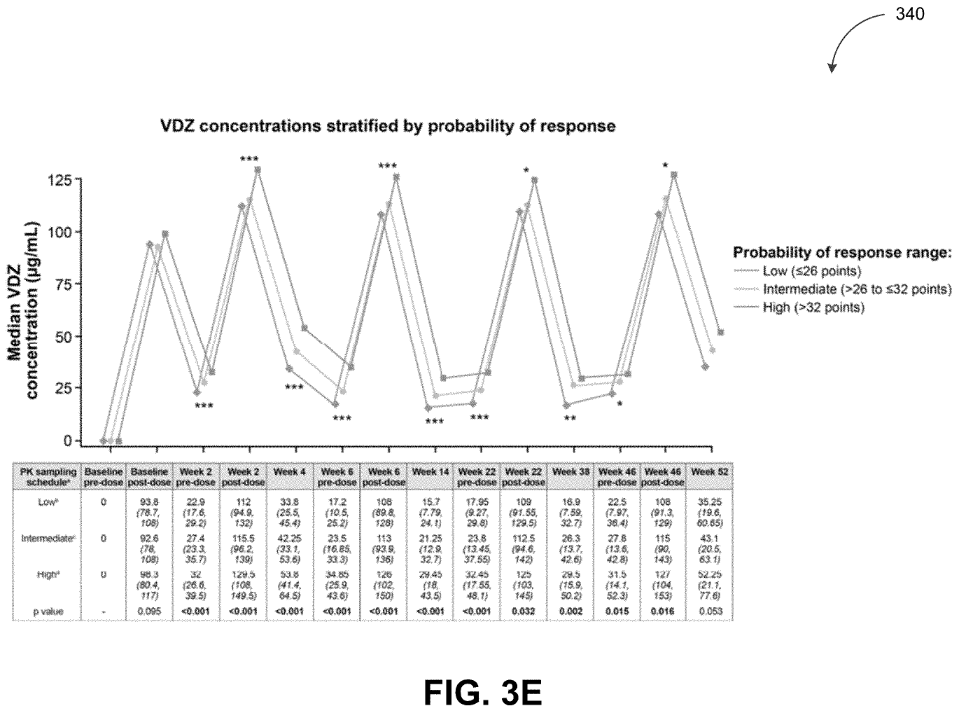

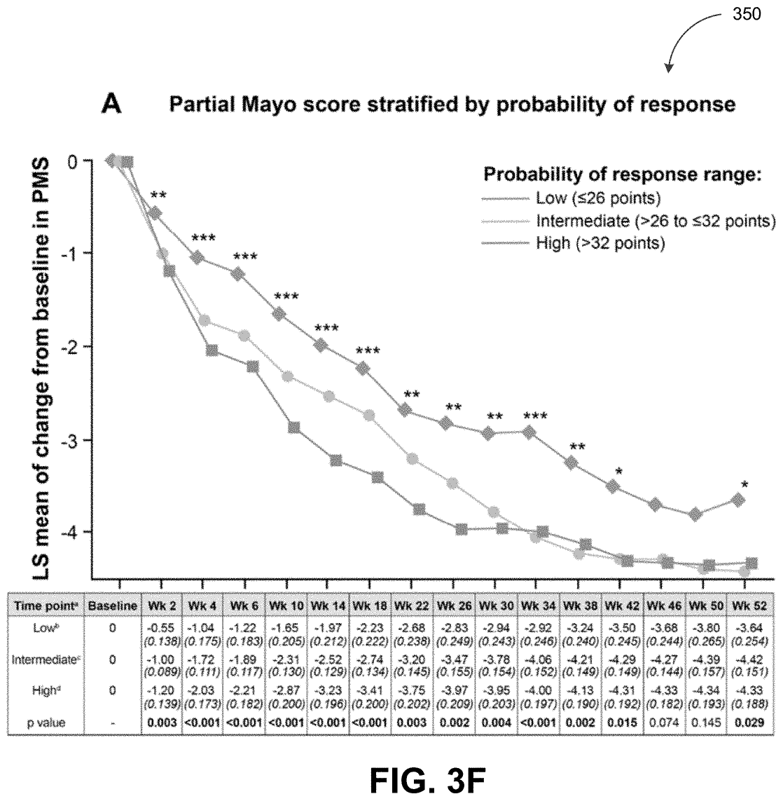

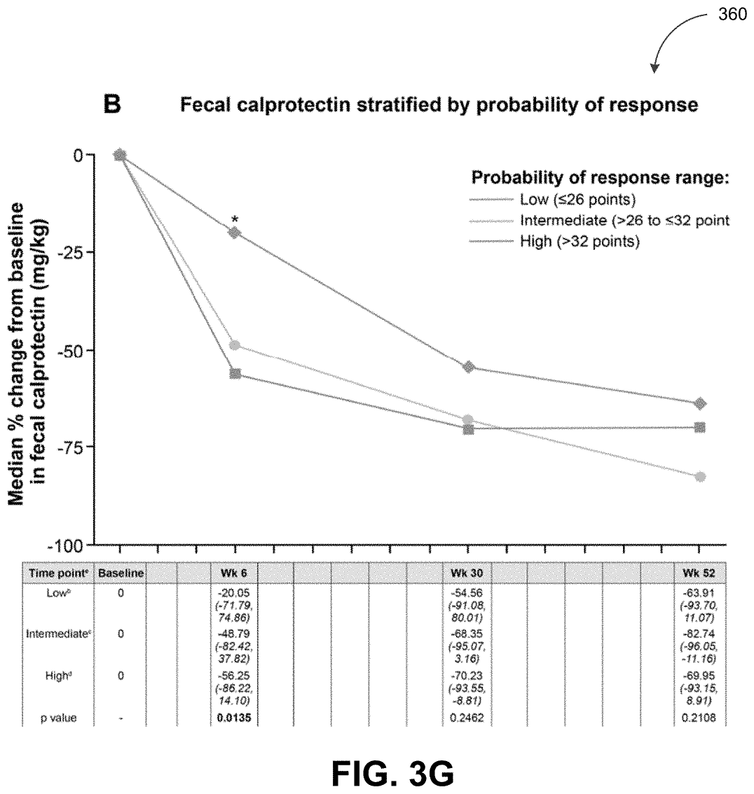

FIG. 2C depicts a table 220 illustrating the diagnostic performance of the predictive model 115, in accordance with some example embodiments. As shown in FIG. 2C, the ability of the predictive model 115 to accurately determine the patient's response to vedolizumab may be evaluated in terms of sensitivity, specificity, positive likelihood ration (PLR), and negative likelihood ratio (NLR). The performance of the predictive model 115 is further shown in FIG. 3A, which depicts a graph 300 comparing actual treatment outcome and the outcome predicted by the predictive model 115. Referring to FIG. 3A, the solid bars in the graph 300 represent outcomes for patients treated with vedolizumab while the striped bars represent outcomes for patients treated with tumor necrosis factor (TNF) antagonist. The outcomes shown in graph 300 include corticosteroid-free clinical and endoscopic remission and subsequent surgical intervention (e.g., colectomy to remove the colon). As shown in FIG. 3A, an increasing probability of response to vedolizumab (e.g., a higher response indicator output by the predictive model) is accompanied by an increasing probability of actually achieving corticosteroid-free clinical and endoscopic remission with vedolizumab as well as a decreasing probability of needing surgical intervention. Graph 300 therefore confirms the ability of the predictive model 115 to predict the outcome for patients treated with vedolizumab. The same observation is not made for tumor necrosis factor (TNF) antagonist.

In some example embodiments, the response indicator I may be further indicative of a likelihood of the patient requiring subsequent surgical intervention (e.g., colectomy to remove the colon) and/or encountering infections during the treatment including the vedolizumab. For example, the predictive model 115 may output a higher response indicator indicative of a higher probability of responding to the treatment including vedolizumab, a higher probability of a rapid response to the treatment including vedolizumab, a higher probability of achieving corticosteroid-free clinical or endoscopic remission, a lower probability of requiring surgical intervention (e.g., colectomy to remove the colon), and/or a lower probability of infection during the treatment including the vedolizumab. Alternatively, the predictive model 115 may output a lower response indicator indicative of a lower probability of responding to the treatment including vedolizumab, a lower probability of a rapid response to the treatment including vedolizumab, a lower probability of achieving corticosteroid-free clinical or endoscopic remission, a higher probability of requiring surgical intervention (e.g., colectomy to remove the colon), and/or a higher probability of infection during the treatment including the vedolizumab. As used herein, a rapid response may refer to a response to vedolizumab that is achieved within a threshold quantity of time. Examples of responses to vedolizumab may include a reduction in the symptoms of ulcerative colitis (e.g., as measured by a Mayo score) and/or a reduction in the biomarkers associated with ulcerative colitis (e.g., fecal calprotectin for colon inflammation and/or the like).

To further illustrate the performance of the predictive model 115, FIG. 3B depicts a table 310 illustrating a performance of the predictive model 115. For example, the table 310 shows the relationship between the output of the predictive model 115 in predicting the probability of an ulcerative colitis patient responding to a treatment including vedolizumab and an actual outcome including subsequent surgical intervention (e.g., colectomy to remove the colon)). Meanwhile FIG. 3C depicts a table 320 that illustrates the performance of the predictive model 115 by showing the relationship between the output of the predictive model 115 in predicting the probability of an ulcerative colitis patient responding to a treatment including vedolizumab and an actual outcome including the occurrence of infections. FIG. 3F depicts a graph 350 illustrating the performance of the predictive model 115 by showing the relationship between the output of the predictive model 115 in predicting the probability of an ulcerative colitis patient responding to a treatment including vedolizumab and an actual outcome including a rapidity of response to the treatment including vedolizumab. FIG. 3G depicts a graph 360 illustrating the performance of the predictive model 115 by showing the relationship between the output of the predictive model 115 in predicting the probability of an ulcerative colitis patient responding to a treatment including vedolizumab and an actual outcome including a reduction in a biomarker (e.g., fecal calprotectin for colon inflammation and/or the like) associated with ulcerative colitis.

In some example embodiments, the treatment controller 110 may determine, based at least on the response indicator output by the predictive model 115, whether the treatment for the ulcerative colitis patient should include vedolizumab. For example, the treatment controller 110 may determine to include vedolizumab in the treatment for the ulcerative colitis patient if the response indicator associated with the patient exceeds a threshold value. That is, the treatment controller 110 may determine to treat the ulcerative colitis patient with vedolizumab if the responds indicator output by the predictive model 115 indicates an above threshold probability of responding to the treatment including vedolizumab, an above threshold probability of achieving corticosteroid-free clinical or endoscopic remission, a below threshold probability of requiring surgical intervention (e.g., colectomy to remove the colon), and/or a below threshold probability of infection during the treatment including the vedolizumab.

In some example embodiments, the treatment controller 110 may be further configured to determine, based on the response indicator associated with the patient, a dose quantity and/or a dose schedule for administering the vedolizumab. The outcome of the vedolizumab treatment may be dependent upon the quantity of drug administered to the patient as well as the length of the interval between administrations of the drug. Notably, patients with higher vedolizumab trough concentrations (e.g., lowest concentration reached by the drug before a subsequent dose) had higher deep remission rates at 52 weeks. Shortening the interval between doses of vedolizumab may be especially effective for patients who are less likely to respond to vedolizumab (e.g., whose response indicator is below a threshold value).

The treatment controller 110 may determine, based at least on the response indicator associated with the patient, a length of an interval between successive administrations of vedolizumab. Alternatively and/or additionally, the treatment controller 110 may identify, based at least on the response indicator associated with the patient, patients who may benefit from an increased exposure to vedolizumab. The relationship between a patient's response to vedolizumab and the concentration of the drug the patient is exposed to is shown in table 330 of FIG. 3D, which depicts the differences in median measured vedolizumab concentrations observed in a patient cohort over the course of 52 weeks. FIG. 3E depicts a graph 340 illustrating the relationship between a patient's response to vedolizumab and the concentration of the drug the patient is exposed to.

Accordingly, if the patient is exposed to a lower concentration of vedolizumab, the treatment controller 110 may determine to increase the quantity of the drug administered to the patient including by increasing the quantity of the drug administered for each dose quantity and/or shortening the interval between doses. Contrastingly, since a patient with a high concentration of vedolizumab tends to derive minimal benefits from additional exposure, the treatment controller 110 may maintain the same dose quantity and/or dose schedule for a patient already exposed to a high concentration of vedolizumab. The treatment controller 110 may send, to the client 130, a treatment plan for the ulcerative colitis patient including the dose quantity and/or the dose schedule for administering the vedolizumab to the patient. The treatment plan may be displayed at the client 130, for example, as part of a user interface associated with the treatment controller 110. In some example embodiments, the ulcerative colitis patient may be treated in accordance with the treatment plan including by being administered vedolizumab at the quantities and/or the intervals set forth in the treatment plan.

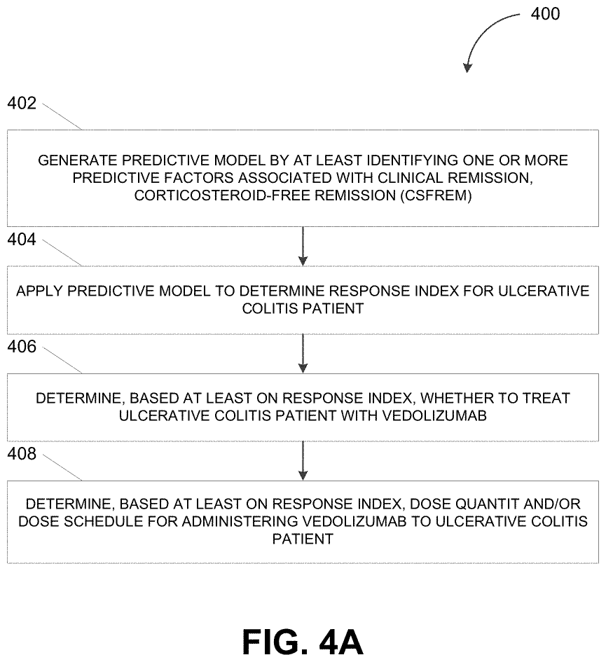

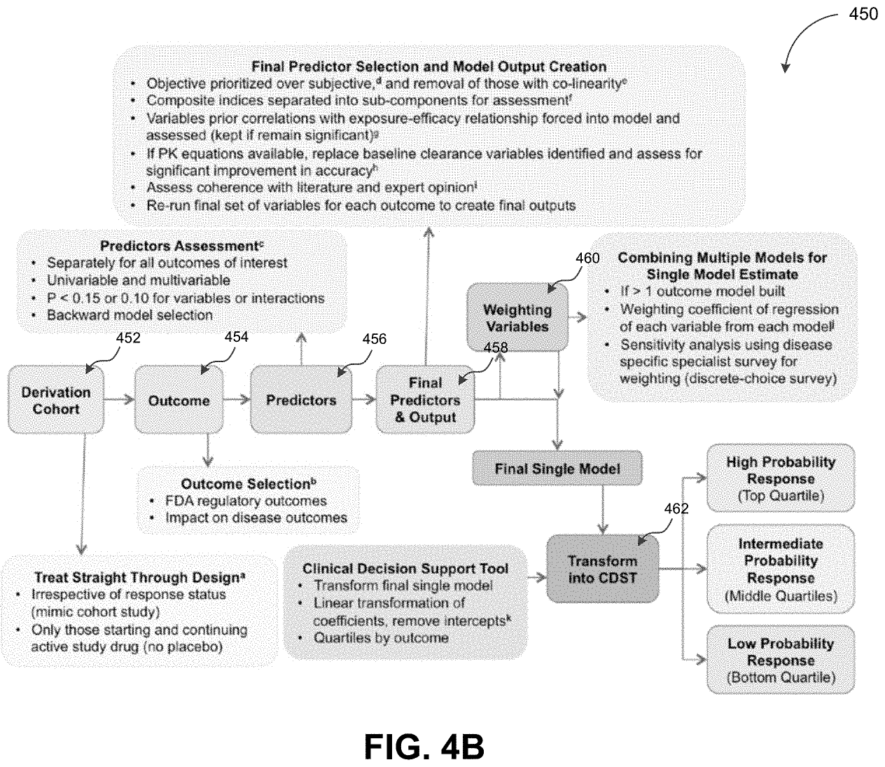

FIG. 4A depicts a flowchart illustrating an example of a process 400 for determining responsiveness to vedolizumab, in accordance with some example embodiments. Referring to FIGS. 1 and 4A, the process 400 may be performed by the treatment controller 110.

At 402, the treatment controller 110 may generate the predictive model 115 by at least identifying one or more predictive factors associated with clinical remission, corticosteroid-free remission (CSFREM). For example, the treatment controller 110 may generate, based on at least a portion of patient data stored in the data store 120, the predictive model 115. The patient data stored in the data store 120 may include one or more observational datasets (e.g., clinical trial datasets and/or the like) associated with ulcerative colitis patients who have been treated with vedolizumab, each of which including measured exposure to vedolizumab, onset of action, and overall efficacy of the vedolizumab treatment. The one or more predictive factors may include disease duration (e.g., ≥2 years and <2 years), previous tumor necrosis factor (TNF) antagonist exposure (e.g., no vs yes), baseline endoscopy (moderate vs severe), and baseline albumin (e.g., absolute value).

At 404, the treatment controller 110 may apply the predictive model 115 to determine a response indicator for an ulcerative colitis patient. For example, the predictive model 115 may output a response indicator indicative of whether a patient treated with vedolizumab will respond to the treatment and achieve clinical remission, corticosteroid-free remission (CSFREM) as well as a likelihood of the patient requiring subsequent surgical intervention (e.g., colectomy to remove the colon) and/or encountering infections during the treatment including the vedolizumab. For example, the predictive model 115 may output a higher response indicator indicative of a higher probability of responding to the treatment including vedolizumab, a higher probability of achieving a rapid response to the treatment including vedolizumab, a higher probability of achieving corticosteroid-free clinical or endoscopic remission, a lower probability of requiring surgical intervention (e.g., colectomy to remove the colon), and/or a lower probability of infection during the treatment including the vedolizumab. Alternatively, the predictive model 115 may output a lower response indicator indicative of a lower probability of responding to the treatment including vedolizumab, a lower probability of achieving a rapid response to the treatment including vedolizumab, a lower probability of achieving corticosteroid-free clinical or endoscopic remission, a higher probability of requiring surgical intervention (e.g., colectomy to remove the colon), and/or a higher probability of infection during the treatment including the vedolizumab.

At 406, the treatment controller 110 may determine, based at least on the response indicator, whether to treat the ulcerative colitis patient with vedolizumab. For example, the treatment controller 110 may determine to include vedolizumab in the treatment for the ulcerative colitis patient if the response indicator associated with the patient exceeds a threshold value. That is, the treatment controller 110 may determine to treat the ulcerative colitis patient with vedolizumab if the responds indicator output by the predictive model 115 indicates an above threshold probability of responding to the treatment including vedolizumab, an above threshold probability of achieving corticosteroid-free clinical or endoscopic remission, a below threshold probability of requiring surgical intervention (e.g., colectomy to remove the colon), and/or a below threshold probability of infection during the treatment including the vedolizumab.

At 408, the treatment controller 110 may determine, based at least on the response indicator, a dose quantity and/or a dose schedule for administering vedolizumab to the ulcerative colitis patient. The outcome of the vedolizumab treatment may be dependent upon the length of the interval between administrations of the drug. For example, shortening the interval between doses of vedolizumab may be especially effective for patients who are less likely to respond to vedolizumab (e.g., whose response indicator is below a threshold value). Accordingly, The treatment controller 110 may determine, based at least on the response indicator associated with the patient, a length of an interval between successive administrations of vedolizumab. Alternatively and/or additionally, the treatment controller 110 may identify, based at least on the response indicator associated with the patient, patients who may benefit from an increased exposure to vedolizumab. The relationship between a patient's response to vedolizumab and the concentration of the drug the patient is exposed to is shown in table 330 of FIG. 3D, which depicts the differences in median measured vedolizumab concentrations observed in a patient cohort over the course of 52 weeks. Accordingly, if the patient is exposed to a lower concentration of vedolizumab, the treatment controller 110 may determine to increase the quantity of the drug administered to the patient including by shortening the interval between doses. Contrastingly, since a patient with a high concentration of vedolizumab tends to derive minimal benefits from additional exposure, the treatment controller 110 may maintain the same dose quantity and/or dose schedule for a patient already exposed to a high concentration of vedolizumab. It should be appreciated that determining the dose quantity and/or dose schedule for administering vedolizumab may include altering an existing dose quantity and/or dose schedule, for example, by increasing the quantity of vedolizumab administered in each dose and/or shortening the interval between successive doses of vedolizumab to increase a patient's exposure to vedolizumab if the patient's response indicator indicates that the patient may benefit from an increased exposure to vedolizumab.

In some example embodiments, the treatment controller 110 may, as noted, generate the predictive model 115 based on at least a portion of the observational datasets (e.g., clinical trial datasets and/or the like) stored in the data store 120. The treatment controller 110 may perform cohort selection in which the patient data stored in the data store 120 may be filtered, for example, to remove patient data that may introduce bias in the subsequent selection of predictive factors including, for example, placebo response, variable response status based on cohort or trial design, and prior exposures or concomitant exposures based on cohort or trial inclusion or a priori stratifications. The resulting ulcerative colitis patient cohorts may be analyzed for each patient's outcome, which may include outcomes used for regulatory approval of vedolizumab as well as interim outcome impacting disease morbidity and/or mortality.

The treatment controller 110 may generate the predictive model 115 by at least selecting one or more predictive factors associated with clinical remission, corticosteroid-free remission (CSFREM). Predictive factors may be selected based on a number of criteria including, for example, objective predictive factors or subjective predictive factors, methods of assessment (e.g, blinded), direct assessments (e.g., endoscopy) and/or surrogate assessments (e.g., biomarkers), subjectivity (e.g., patient or provider), and/or the like. When co-linearity is observed, the treatment controller 110 may retain, as a predictive factor in the predictive model 115, the variable that is most easily measured and widely available, and most closely associated with disease or treatment outcome. When a composite disease activity indicator meets the thresholds for potential inclusion in the predictive model 115, the sub-components of that indicator may be assessed individually to determine which sub-score is driving the significance of association, what component or value within that sub-score is most significant, and if that sub-score maintains a linear trend in association with higher/lower values or if it is a dichotomous association. For example, albumin, C-reactive protein, weight, height, gender, and body mass indicator (BMI) may be assessed based on associations with variations in exposure across diseases and drugs. However, a variable (e.g., weight) may be removed from the predictive model due to co-linearity with other variables. Pharmacokinetic (PK) equations may be taken from population pharmacokinetic studies, exposure-efficacy studies, and/or variations of such studies. Once a final predictive model 115 is identified, and a pharmacokinetic equation is available, all baseline variables associated with regulatory clearance (i.e. albumin) may be removed and the pharmacokinetic equation may be selectively added to assess for changes in the accuracy or consistency in the performance of predictive model 115.

For at least some predictive factors identified by the treatment controller 110, subgroup analyses stratified by the predictive factors may be performed in order to assess for congruence and comparisons in distribution to other predictive factors potentially driving significance. When derivation models are created for >1 outcome, the treatment controller 110 may generate a single final predictive model 115, the treatment controller 110 may perform a weighting of the coefficient of regression of each variable from each individual model by an estimation of the inverse variance for each coefficient estimate. For example, the weighting of the coefficient of regression may be performed by multiplying the β regression coefficient of predictive factors in the predictive model 115 by a numerical factor (e.g., 10) and removing the intercept.

To further illustrate, FIG. 4B depicts a flowchart illustrating an example of a process 450 for generating the predictive model 115, in accordance with some example embodiments. Referring to FIGS. 1 and 4A-B, the process 450 may be performed by the treatment controller 110. In some example embodiments, the process 450 may implement operation 402 of the process 400 shown in FIG. 4A.

At 452, the treatment controller 110 may generate an ulcerative colitis patient cohort by removing, from observational datasets (e.g., clinical trial datasets stored in the data store 120), data associated with participants who were exposed to a placebo or non-intervention. Accordingly, the result patient cohort may include clinical trial participants who are treated with the active agent (e.g., vedolizumab).

At 454, the treatment controller 110 may identify the outcome to be predicted by the predictive model 115 including, for example, exposure to vedolizumab and the efficacy of vedolizumab.

At 456, the treatment controller 110 may identify one or more predictive factors for inclusion in the predictive model 115. For example, the treatment controller 110 may select predictive factors from one or more pre-defined characteristics such as patient characteristics (e.g., age, gender, body mass indicator, height, weight, smoking history, and/or the like), disease characteristics (e.g., disease subgroup, disease duration, prior treatments (e.g., tumor necrosis factor, immunomodulatory, steroids), prior hospitalization, disease progression (e.g., limited to the rectum and the rectosigmoid colon or extend beyond the splenic flexure), and/or the like), lab characteristics (e.g., C-reactive protein concentration and albumin concentration prior to vedolizumab exposure), and disease severity (e.g., clinical symptoms (e.g., Mayo score), endoscopic activity (e.g., Mayo endoscopic sub-score)).

In some example embodiments, the treatment controller 110 may assess each predictive factor individually, for example, using logistic regression, to identify predictive factors that meet a threshold of inclusion (e.g., P value of 0.15) for predicting responses to a treatment including vedolizumab. Logistic regression may be performed on the predictive factors that meet the inclusion threshold in order to screen and remove predictive factors that yield competing predictions and/or exhibit collinearity. The remaining predictive factors may be further assessed for interactions with each other for predicting responses to vedolizumab using a more stringent threshold (e.g., P value of 0.10).

At 458, the treatment controller 110 may generate the predictive model 115 by weighing the predictive factors to combine the predictive factors into a single model. For example, the treatment controller 110 may generate the predictive model 115 to include one or more patient characteristics, disease characteristics, lab characteristics, and disease severity characteristics. Multiple predictive factors may be combined into a single predictive model 115 by weighting each predictive factor and assigning a point scoring system to each individual predictive factor to form a single linear score, for example, a response indicator, indicative of a patient's response to a treatment including vedolizumab. For instance, as noted, the final included set of predictors which may include patient characteristics, disease characteristics, lab characteristics, or disease severity features, are then combined into a single tool by weighting each predictor and assigning a point scoring system to each individual predictor which can be combined together to create a linear score.

At 460, the treatment controller 110 may separate the outputs of the predictive model 115 into one or more groups representative of the various probability of responding to vedolizumab. For example, the treatment controller 110 may separate, based at least on the response indicator associated with each patient in the cohort, the cohort into three groups including a first group of patients having a high probability of response to vedolizumab (e.g., patients in the top 25% of the response indicator range), a second group of patients having an intermediate probability of response to vedolizumab (e.g., patients in the middle 50% of the response indicator range), and a third group of patients having a low probability of response to vedolizumab (e.g., patients in the bottom 25% of the response indicator range).

At 462, the treatment controller 110 may assess an ability of the predictive model 115 to predict the outcome of a treatment including vedolizumab. For example, the performance of the predictive model 115 may be assessed by determining how accurately the predictive model 115 is able to predict the treatment outcome of patients in a separate cohort than the one used to generate the predictive model 115.

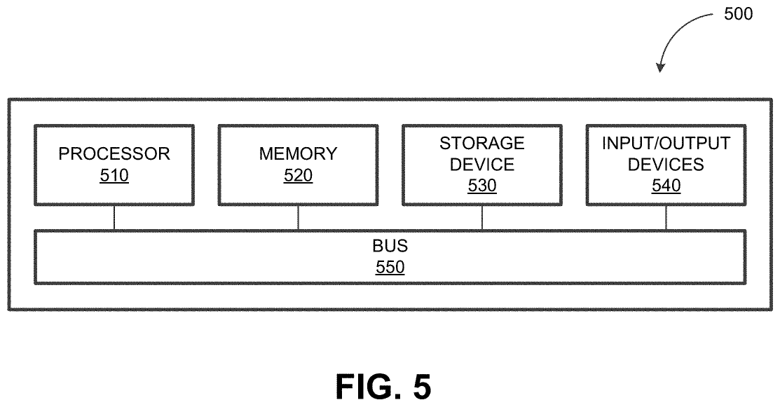

FIG. 5 depicts a block diagram illustrating a computing system 500 consistent with implementations of the current subject matter. Referring to FIGS. 1 and 5, the computing system 500 can be used to implement the treatment controller 110 and/or any components therein.

As shown in FIG. 5, the computing system 500 can include a processor 510, a memory 520, a storage device 530, and an input/output device 540. The processor 510, the memory 520, the storage device 530, and the input/output device 540 can be interconnected via a system bus 550. The processor 510 is capable of processing instructions for execution within the computing system 500. Such executed instructions can implement one or more components of, for example, the mass spectrometer 110, the processing engine 120, the analysis controller 130, the client 140. In some example embodiments, the processor 510 can be a single-threaded processor. Alternately, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, a tape device, a solid-state device, and/or other suitable persistent storage means. The input/output device 540 provides input/output operations for the computing system 500. In some example embodiments, the input/output device 540 includes a keyboard and/or pointing device. In various implementations, the input/output device 540 includes a display unit for displaying graphical user interfaces.

According to some example embodiments, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example embodiments, the computing system 500 can be used to execute various interactive computer software applications that can be used for organization, analysis and/or storage of data in various formats. Alternatively, the computing system 500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively, or additionally, store such machine instructions in a transient manner, such as for example, as would a processor cache or other random access memory associated with one or more physical processor cores.

What is claimed is:

1. A system, comprising:
at least one data processor; and
at least one memory storing instructions, which when executed by the at least one data processor, result in operations comprising:
applying a predictive model to determine a response indicator indicative of the outcome of treating the ulcerative colitis patient with vedolizumab, the response indicator including a first probability of the ulcerative colitis patient responding to vedolizumab, the predictive model based on a plurality of predictive factors including a duration of disease; and
determining, based at least on the response indicator, a treatment plan for the ulcerative colitis patient.

2. The system of claim 1, wherein the response indicator further includes a second probability of the ulcerative colitis patient achieving a clinical remission and/or an endoscopic remission of ulcerative colitis with vedolizumab, a third probability of the ulcerative colitis patient achieving a rapid response to vedolizumab, a fourth probability of the ulcerative colitis patient requiring surgical intervention, and/or a fifth probability of infection when treated with vedolizumab.

3. The system of claim 1, wherein the plurality of predictive factors further include an exposure to tumor necrosis factor antagonist therapy.

4. The system of claim 1, wherein the plurality of predictive factors further include a baseline endoscopy corresponding to a disease severity.

5. The system of claim 1, wherein the plurality of predictive factors further include a concentration of albumin.

6. The system of claim 1, wherein the duration of disease comprises a first value to indicate a duration that is less than a quantity of time or a second value to indicate a duration that is equal to or greater than the quantity of time.

7. The system of claim 1, further comprising:
generating the predictive model including by identifying, based on one or more observational datasets associated with vedolizumab, the plurality of predictive factors, the identifying of the plurality of predictive factors includes generating a first cohort of ulcerative colitis patients, and the first cohort of ulcerative colitis patients being generated by at least removing, from the one or more observational datasets, data associated with ulcerative colitis patients exposed to a placebo instead of vedolizumab.

8. The system of claim 7, wherein the generating of the predictive model further includes generating a second cohort of ulcerative colitis patients, and wherein a performance of the predictive model is verified based at least on data associated with the second cohort of ulcerative colitis patients.

9. The system of claim 7, wherein the generating of the predictive model further includes excluding, from the predictive model, a first predictive factor that exhibits a co-linearity with a second predictive factor.

10. The system of claim 7, wherein the generating of the predictive model further includes determining, for each of the plurality of predictive factors, a weight corresponding to a correlation to a treatment outcome of vedolizumab.

11. The system of claim 7, wherein the plurality of predictive factors are identified by performing a logistic regression on the one or more observational datasets to identify the plurality of predictive factors as having an above-threshold correlation with a treatment outcome of vedolizumab.

12. The system of claim 1, wherein the treatment plan for the ulcerative colitis patient includes vedolizumab in response to the response indicator exceeding a threshold value.

13. The system of claim 11, wherein the treatment plan for the ulcerative colitis patient further includes a dose quantity comprising a quantity of vedolizumab administered in each dose of vedolizumab.

14. The system of claim 12, wherein the treatment plan for the ulcerative colitis patient further includes a dose schedule for administering vedolizumab, and wherein the dose schedule includes a length of an interval between successive doses of vedolizumab.

15. The system of claim 13, wherein the length of the interval between successive doses of vedolizumab corresponds to the response indicator associated with the ulcerative colitis patient.

16. The system of claim 13, wherein the ulcerative patient is treated in accordance with the treatment plan including by being administered the quantity of vedolizumab at one or more intervals indicated by the dose schedule.

17. A computer-implemented method, comprising:
applying a predictive model to determine a response indicator indicative of the outcome of treating the ulcerative colitis patient with vedolizumab, the response indicator including a first probability of the ulcerative colitis patient responding to vedolizumab, the predictive model based on a plurality of predictive factors including a duration of disease; and
determining, based at least on the response indicator, a treatment plan for the ulcerative colitis patient.

18. The computer-implemented method of claim 17, wherein the response indicator further includes a second probability of the ulcerative colitis patient achieving a clinical remission and/or an endoscopic remission of ulcerative colitis with vedolizumab, a third probability of the ulcerative colitis patient achieving a rapid response to vedolizumab, a fourth probability of the ulcerative colitis patient requiring surgical intervention, and/or a fifth probability of infection when treated with vedolizumab.

19. The computer-implemented method of claim 17, wherein the plurality of predictive factors further include an exposure to tumor necrosis factor antagonist therapy, a baseline endoscopy corresponding to a disease severity, and/or a concentration of albumin.

20. A non-transitory computer readable medium storing instructions, which when executed by at least one data processor, result in operations comprising:
applying a predictive model to determine a response indicator indicative of the outcome of treating the ulcerative colitis patient with vedolizumab, the response indicator including a first probability of the ulcerative colitis patient responding to vedolizumab, the predictive model based on a plurality of predictive factors including a duration of disease; and
determining, based at least on the response indicator, a treatment plan for the ulcerative colitis patient.
